(19) United States
(12) Patent Application Publication
(10) Pub. No.: US 2026/0151107 A1
(43) Pub. Date: Jun. 4, 2026

(54) CABLE MANAGEMENT APPARATUS FOR MEDICAL DEVICE AND MEDICAL DEVICE

(71) Applicant: GE Precision Healthcare LLC, Waukesha, WI (US)

(72) Inventors: Yalan Yang, Wuxi (CN); Xinyu Bian, Wuxi (CN); Jinshan Wang, Wuxi (CN)

(21) Appl. No.: 19/394,603

(22) Filed: Nov. 19, 2025

(30) Foreign Application Priority Data
Nov. 29, 2024 (CN) 202411745119.1

(51) Int. Cl. A61B 8/00; A61B 50/20

(52) U.S. Cl. CPC A61B 8/4405; A61B 50/20

(57) ABSTRACT

A cable management apparatus for a medical device, and the medical device. The cable management apparatus includes: a restraint portion, having a through hole capable of receiving at least part of a cable of the medical device; and a driving portion, connected to the restraint portion and configured to be triggered to: control the restraint portion to move along a first direction to restrain the at least part of the cable entering the through hole, and/or control the restraint portion to move along a second direction opposite to the first direction. According to the embodiments of the present application, the cable can be automatically arranged, thereby omitting the step of manual cable management, so that not only work efficiency can be improved, but user experience can also be improved.

### CROSS-REFERENCE TO RELATED APPLICATION

This application claim priority to Chinese Patent Application No. 202411745119.1, which was file on Nov. 29, 2024 at the Chinese Patent Office. The entire contents of the above-listed application are incorporated by reference herein in their entirety.

### TECHNICAL FIELD

Embodiments of the present application relate to the field of medicine, and in particular, to a cable management apparatus for a medical device, and the medical device.

### BACKGROUND

In an existing medical device, a probe is connected to a connector by means of a cable, and the connector is fixed to a main unit and electrically connected to the main unit. During scanning, a doctor holds the probe and moves the probe back and forth within a scanning area, and image information acquired by the probe is transmitted to the main unit by means of the cable and the connector.

For example, for a medical ultrasonic device, one medical ultrasonic device is usually equipped with one or more ultrasonic probes, and each probe is connected to an ultrasonic main unit by means of a corresponding probe cable and probe connector. Most of the current medical ultrasonic devices use a lifting console, and in order to adapt to the adjustable height of the lifting console, the probe cable is usually designed with a large margin in the length, resulting in a long cable length, for example, 1.8 to 2 meters. If the probe cables are not managed, the probes and cables are scattered and twisted together when not in use. Therefore, the cables are apt to be crossed and entangled, which may cause maloperations.

In addition, since the root portion of a probe is fragile, the root portion usually requires protection and management. Management of probes and cables is important for the efficiency and safety of use of medical devices. Therefore, in some ultrasonic devices, cable management apparatuses are used to manage cables of ultrasonic probes.

### SUMMARY OF THE INVENTION

However, it has been found that: by using the existing cable management apparatus, a cable slot needs to be located manually, and a cable insertion operation and a cable management operation are performed separately. Therefore, a user needs to manage cables manually. The operation is not simple and convenient enough and increases the workload of the user.

In view of at least one of the above technical problems, the embodiments of the present application provide a cable management apparatus for a medical device, and the medical device.

According to an aspect of the embodiments of the present application, a cable management apparatus for a medical device is provided. The cable management apparatus comprises:
a restraint portion, having a through hole capable of receiving at least part of a cable of the medical device; and
a driving portion, connected to the restraint portion and configured to be triggered to: control the restraint portion to move along a first direction to restrain the at least part of the cable entering the through hole, and/or control the restraint portion to move along a second direction opposite to the first direction.

In some embodiments, the through hole passes through the restraint portion along the first direction and the second direction; and the restraint portion is further provided with an opening facing a third direction, the at least part of the cable enters or leaves the through hole by means of the opening, and the third direction is perpendicular to the first direction and the second direction.

In some embodiments, the opening has a size smaller than the diameter of the cable, and the cable enters the through hole or leaves the through hole via the opening by means of elastic deformation of the restraint portion.

In some embodiments, the restraint portion has a clamping structure, the clamping structure comprises a first elastic sheet and a second elastic sheet disposed oppositely, and the through hole and the opening are formed between the first elastic sheet and the second elastic sheet.

In some embodiments, the driving portion comprises:
a motor, configured to drive the restraint portion to move from a first position to a second position along the first direction when receiving a first trigger command, and/or drive the restraint portion to move from the second position to the first position along the second direction when receiving a second trigger command.

In some embodiments, the cable management apparatus further comprises:
a trigger portion, configured to generate the first trigger command when the at least part of the cable enters the through hole and generate the second trigger command when the at least part of the cable leaves the through hole.

According to another aspect of the embodiments of the present application, a medical device is provided. The medical device comprises:
a probe, which comprises a transducer, a cable, and a connector, the cable connecting the transducer and the connector;
a base, which comprises a probe socket, the probe socket being capable of receiving the connector; and
the cable management apparatus as previously described.

In some embodiments, a restraint portion of the cable management apparatus is disposed on the base, the probe socket of the base and the restraint portion are provided along a first direction, and the distance between the probe socket and the restraint portion corresponds to the length of the connector, to enable the cable at the tail end of the connector to correspond to the restraint portion when the connector is inserted into the probe socket.

In some embodiments, a driving portion of the cable management apparatus is disposed on the base, and the base is further provided with a slot portion for the restraint portion of the cable management apparatus to move along a first direction or a second direction.

In some embodiments, the base is further provided with a locking switch for locking the connector; and the locking switch further serves as a trigger portion for triggering a driving portion of the cable management apparatus.

In some embodiments, when the locking switch is in a locked position, the connector is locked within the probe socket, and the driving portion is triggered to control the restraint portion to move along a first direction, and/or, when the locking switch is in an unlocked position, the connector is unlocked from the probe socket, and the driving portion is triggered to control the restraint portion to move along a second direction.

In some embodiments, a trigger portion of the cable management apparatus is disposed on the restraint portion.

In some embodiments, when at least part of the cable enters the through hole of the restraint portion, the driving portion is triggered to control the restraint portion to move along a first direction, and/or, when at least part of the cable leaves the through hole of the restraint portion, the driving portion is triggered to control the restraint portion to move along a second direction.

In some embodiments, a trigger portion of the cable management apparatus is disposed in the probe socket.

In some embodiments, when the connector is inserted into the probe socket, the driving portion is triggered to control the restraint portion to move along a first direction, and/or, when the connector is pulled out from the probe socket, the driving portion is triggered to control the restraint portion to move along a second direction.

Beneficial effects of the embodiments of the present application are as follows: the driving portion of the cable management apparatus controls the restraint portion to move along a first direction to restrain the at least part of the cable entering the through hole, and/or controls the restraint portion to move along a second direction opposite to the first direction. Therefore, the cable can be automatically arranged, thereby omitting the step of manual cable management, so that not only work efficiency can be improved, but user experience can also be improved.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a medical device according to an embodiment of the present application;

FIG. 2 is a schematic diagram of a cable management apparatus according to an embodiment of the present application;

FIG. 3 is a schematic diagram of a restraint portion according to an embodiment of the present application;

FIG. 4 is a schematic diagram of a cable before being inserted into the restraint portion according to an embodiment of the present application;

FIG. 5 is a schematic diagram of a cable after being inserted into the restraint portion according to an embodiment of the present application;

FIG. 6 is a schematic diagram after cable management according to an embodiment of the present application;

FIG. 7 is a schematic diagram of a motor according to an embodiment of the present application;

FIG. 8 is a schematic cross-sectional view of a motor and a restraint portion according to an embodiment of the present application; and

FIG. 9 is a schematic diagram of part of a base according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc. are used to distinguish different elements from one another by title, but do not represent the spatial arrangement, temporal order, etc. of the elements, and the elements should not be limited by said terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies. The terms “connect”, “connected”, “couple”, as well as other similar terms to which the embodiments of the present application relate are not limited to physical or mechanical connections, but may include electrical connections, whether directly connected or indirectly connected.

In the embodiments of the present application, the singular forms “a”, “the”, etc. include plural forms, and should be broadly construed as “a type of” or “a class of” rather than being limited to the meaning of “one”. Furthermore, the term “the” should be construed as including both the singular and plural forms, unless otherwise explicitly specified in the context. In addition, the term “according to” should be construed as “at least in part according to . . . ”, and the term “based on” should be construed as “at least in part based on . . . ”, unless otherwise explicitly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

In the embodiments of the present application, the medical device at least includes: a probe, a base, and a cable management apparatus. The probe includes a transducer, a cable, and a connector, and the cable connects the transducer and the connector. The base includes a probe socket, and the probe socket is capable of receiving the connector.

For example, an ultrasonic device, an ultrasonic probe, and an ultrasonic probe cable are used to describe the cable management apparatus and the medical device in the present application below. Those skilled in the art would appreciate that the present application can also be applied to other medical devices requiring cable management. The embodiments of the present application are not limited thereto.

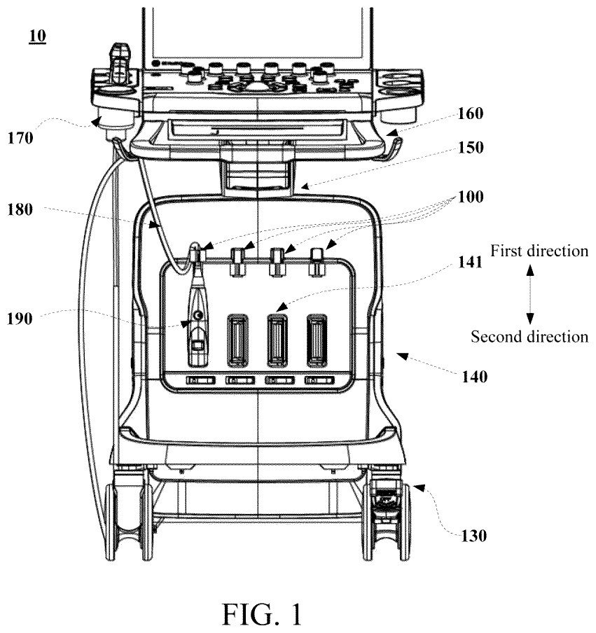

FIG. 1 is a schematic diagram of a medical device according to an embodiment of the present application Exemplarily, an ultrasonic device 10 having an ultrasonic probe cable management apparatus (hereinafter, may be referred to as a cable management apparatus for short) 100 is shown. The ultrasonic device 10 includes a chassis 130 having casters, a base (an ultrasonic main unit) 140 mounted on the base 130, a console 160 connected to the base 140 by means of a lifting connection structure 150, a transducer (an ultrasonic probe) 170 that can be removed and secured to the console 160, and a cable 180 and a connector 190 for electrically connecting the transducer 170 to the base 140.

As shown in FIG. 1, the cable 180 electrically connects the transducer 170 and the connector 190. The base 140 is provided with at least one (four are shown in FIG. 1) probe socket (a connector port) 141, and the connector 190 can be docked with any one of the probe sockets 141, so that the transducer 170 connected to the connector 190 is electrically connected to the base 140. The cable management apparatus 100 will be described in more detail below, and other parts in the ultrasonic device 10 may adopt any structure and arrangement known in the art, which is not repeated herein.

Although only one ultrasonic probe is schematically shown in FIG. 1, practically, one ultrasonic device 10 may be equipped with a plurality of ultrasonic probes. For example, the ultrasonic device 10 in FIG. 1 may be equipped with four ultrasonic probes corresponding to the four probe sockets 141, and the four ultrasonic probes are connected to the four probe sockets 141 by means of corresponding cables and connectors, respectively. The number of ultrasonic probes or probe sockets may not be limited, and different numbers of ultrasonic probes or probe sockets may be provided as required in different embodiments.

As shown in FIG. 1, the cable management apparatus 100 is disposed on the base 140. The cable management apparatus 100 may be integrally formed with the base 140, or the cable management apparatus 100 may alternatively be a separate component and mounted on the base 140; the present application is not limited thereto. The cable management apparatus 100 may include one or more cable receiving structures (hereinafter, referred to as restraint portions). For example, one of the cable receiving structures is used for description below, and the other cable receiving structures may have the same or similar configuration.

In some embodiments, the number of cable receiving structures may correspond to the number of probe sockets. For example, in the ultrasonic device 10 shown in FIG. 1, four cable receiving structures arranged in parallel are provided corresponding to the four probe sockets 141. The number of cable receiving structures in the cable management apparatus is not limited, and different numbers of cable receiving structures may be provided as required in different embodiments.

In the embodiments of the present application, the upward direction in FIG. 1 is referred to as the “first direction”, the downward direction in FIG. 1 is referred to as the “second direction”, and the direction away from the paper surface in FIG. 1 is referred to as the “third direction”. It is worth noting that the definition of each direction in the present description is merely for the convenience of illustrating the embodiments of the present application and does not define the direction of the medical device, the cable management apparatus, etc. when used and manufactured.

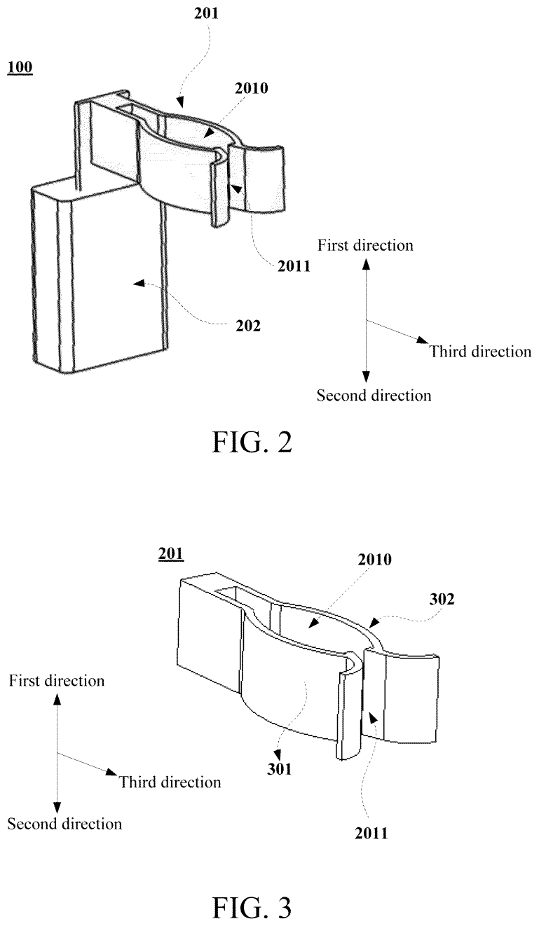

FIG. 2 is a schematic diagram of a cable management apparatus 100 according to an embodiment of the present application. As shown in FIG. 2, the cable management apparatus 100 may be a separate component and may include a restraint portion 201 and a driving portion 202. The cable management apparatus 100 may be part of the base 140.

As shown in FIG. 2, the restraint portion 201 is provided with a through hole 2010 capable of receiving at least part of the cable 180, and the driving portion 202 is connected to the restraint portion 201 and can be triggered to: control the restraint portion 201 to move along a first direction to restrain the at least part of the cable 180 entering the through hole 2010 (which is omitted in FIG. 2 and may be referred to in FIG. 1), and/or, control the restraint portion 201 to move along a second direction opposite to the first direction.

Referring to FIG. 1 and FIG. 2, for example, when the cable 180 enters the restraint portion 201, the driving portion 202 controls the restraint portion 201 to move along the first direction, so that the restraint portion 201 moves upward (away from the probe socket 141) by a distance, and in this process, the cable 180 can be straightened without manual arrangement, thereby completing cable management. When the cable 180 leaves the restraint portion 201, the driving portion 202 controls the restraint portion 201 to move along the second direction, so that the restraint portion 201 moves downward (close to the probe socket 141) by a distance to return to an idle state of the cable management apparatus 100, thereby facilitating re-entry of the cable into the restraint portion 201 when the probe is subsequently placed.

Therefore, the cable can be automatically arranged, so that the step of manual cable management can be omitted.

In some embodiments, the through hole 2010 passes through the restraint portion 201 along the first direction and the second direction. The restraint portion 201 is further provided with an opening 2011 facing a third direction, and the at least part of the cable 180 enters or leaves the through hole 2010 by means of the opening 2011.

FIG. 3 is a schematic diagram of a restraint portion according to an embodiment of the present application. The through hole 2010 has an opening 2011 on one side thereof, and therefore, the cable 180 can enter the through hole 2010 when the connector 190 is inserted into the base 140. That is, when the connector 190 is inserted into the probe socket 141, the cable 180 at the tail portion of the connector 190 can enter the through hole 2010 via the opening 2011, so that two operations of probe insertion and cable arrangement are implemented in one step, thereby further simplifying the operation.

As shown in FIG. 3, the opening 2011 may be flared, so that the cable can be inserted more conveniently, but the present application is not limited thereto, for example, the opening may be in any shape suitable for cable insertion. The opening 2011 may have a size slightly smaller than the diameter of the cable 180, and the through hole 2010 may have a size slightly greater than the diameter of the cable 180. The cable 180 enters the through hole 2010 or leaves the through hole 2010 via the opening 2011 by means of elastic deformation of the restraint portion 201, thereby further simplifying the operation.

For example, as shown in FIG. 3, the restraint portion 201 may include a sheet clamp, so as to restrict the position of the cable. That is, the restraint portion 201 has a clamping structure, the clamping structure includes a first elastic sheet 301 and a second elastic sheet 302 disposed oppositely, and the through hole 2010 and the opening 2011 are formed between the first elastic sheet 301 and the second elastic sheet 302. By means of the clamping structure formed by the elastic sheets, cable management restraint can be achieved with a simple structure.

FIG. 2 and FIG. 3 exemplarily illustrate the restraint portion 201 of the cable management apparatus 100 in the embodiments of the present application, but the present application is not limited thereto. For example, any structure that can restraint a cable and move same by a certain distance for cable management should fall within the scope of the embodiments of the present application.

In some embodiments, a first length of the clamping structure in the third direction corresponds to (for example, equal or substantially equal to) a second length of the connector 190 at the tail end of the cable in the third direction after being inserted into the base 140. The first length and the second length are approximately the same, thereby further ensuring that the cable is held in place after the connector 190 is inserted into the base 140.

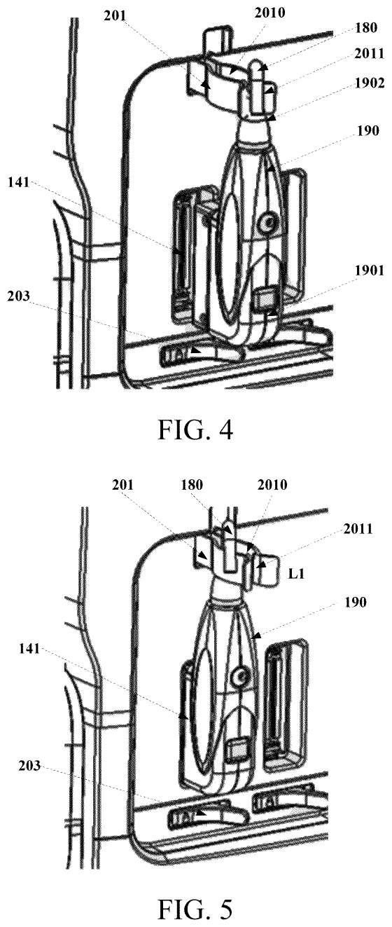

FIG. 4 is a schematic diagram of a cable before being inserted into the restraint portion according to an embodiment of the present application. As shown in FIG. 4, in this case, the connector 190 has not yet been inserted into the probe socket 141, and the cable 180 has not yet entered the restraint portion 201. In this case, a front end 1901 of the connector 190 is close to the probe socket 141, and a tail portion 1902 of the connector 190 is close to the opening 2011 of the restraint portion 201.

FIG. 5 is a schematic diagram of a cable after being inserted into the restraint portion according to an embodiment of the present application. As shown in FIG. 5, in this case, the connector 190 is inserted into the probe socket 141, and the cable 180 enters the through hole 2010 of the restraint portion 201 via the opening 2011. In this case, the restraint portion 201 has not yet been triggered and is located at a first position L1.

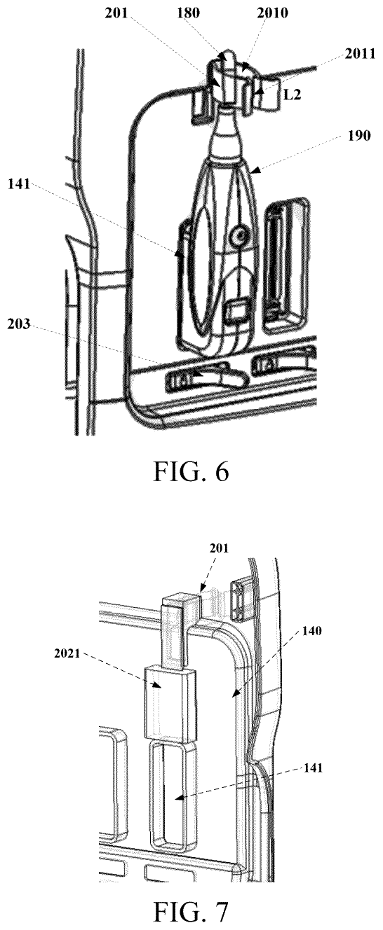

FIG. 6 is a schematic diagram after cable management according to an embodiment of the present application. As shown in FIG. 6, the connector 190 is inserted into the probe socket 141, and the cable 180 enters the through hole 2010 of the restraint portion 201 via the opening 2011. In this case, the restraint portion 201 has already been triggered by the trigger portion, and is located at a second position L2 after moving upward by a certain distance.

The second position L2 is away from the probe socket 141 by a certain distance relative to the first position L1. The distance may be preset. For example, the restraint portion 201 moves within a groove on the base 140, and the length of the groove is the distance by which the restraint portion 201 can move.

FIGS. 4 to 6 exemplarily illustrate that the restraint portion automatically performs cable management, but the present application is not limited thereto. The driving portion and the trigger portion of the cable management apparatus will be described below.

In some embodiments, the driving portion 202 includes a motor. The motor drives the restraint portion 201 to move from a first position to a second position along the first direction when receiving a first trigger command (for example, an ON signal), and/or drives the restraint portion 201 to move from the second position to the first position along the second direction when receiving a second trigger command (for example, an OFF signal).

FIG. 7 is a schematic diagram of a motor according to an embodiment of the present application. For example, FIG. 7 exemplarily illustrates a case in which a motor 2021 in the driving portion 202 is mounted on the base. As shown in FIG. 7, the motor 2021 may be located inside the base 140, to control the restraint portion 201 to move along the first direction or/and the second direction. The specific mounting position of the motor 2021 may be determined according to the specific structure of the medical device, which is not limited in the present application.

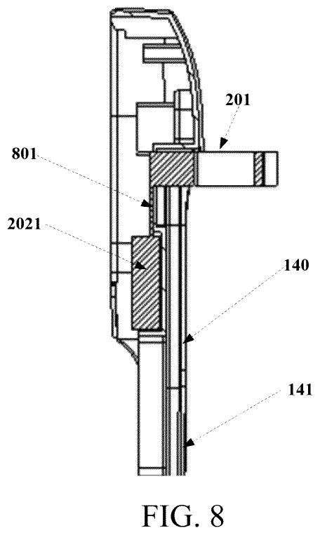

FIG. 8 is a schematic cross-sectional view of a motor and a restraint portion according to an embodiment of the present application, and shows a partial cross-sectional view of the cable management apparatus 100 mounted on the base 140 observed from one side.

For example, the motor 2021 may be a linear motor (for example, also referred to as a linear motor or a push rod motor), and may directly convert electrical energy into mechanical energy of linear motion, for example, drive the restraint portion 201 to move linearly in the up-down direction by means of a push rod 801 or the like, so as to implement movement of the restraint portion 201 between the first position and the second position.

The driving portion 202 of the present application is not limited to a linear motor, and for example, driving of the restraint portion may alternatively be implemented by means of a magnetic induction apparatus or the like, which is not limited in the present application. In addition, the driving portion 202 may drive only one restraint portion 201, that is, the driving portion 202 and the restraint portion 201 are in one-to-one correspondence to achieve separate driving, or the driving portion 202 may also simultaneously control a plurality of restraint portions 201, that is, the driving portion 202 and the restraint portions 201 are in one-to-many correspondence to achieve centralized driving.

In some embodiments, the driving portion is controlled by the trigger portion, and the cable management apparatus further includes: a trigger portion, configured to generate a first trigger command when the at least part of the cable enters the through hole and generate a second trigger command when the at least part of the cable leaves the through hole.

For example, the trigger portion may be disposed in the cable management apparatus, or may be disposed on the base. In FIGS. 4-6, for example, for a locking switch 203, and the locking switch 203 may serve as the trigger portion to send a trigger command. For example, the locking switch 203 sends a first trigger command to the driving portion 202 when locking the connector 190, and the locking switch 203 sends a second trigger command to the driving portion when unlocking the connector 190. In the examples of FIGS. 4-6, the position of the locking switch 203 is located on the base, but the present application is not limited thereto, for example, in other embodiments, the position of the locking switch may be located on another position, for example, on the connector. In addition, the trigger portion may not be provided on the locking switch 203, and further details of the trigger portion may refer to the following embodiments.

The cable management apparatus of the embodiments of the present application is schematically illustrated above, and the medical device will be schematically illustrated below.

An embodiment of the present application provides a medical device, which may refer to FIG. 1, and the same content as in the previous embodiments is not described again herein.

In some embodiments, the restraint portion 201 of the cable management apparatus 100 is disposed or mounted on the base 140, and the probe socket 141 of the base 140 and the restraint portion 201 are disposed along the first direction. The distance between the probe socket 141 and the restraint portion 201 corresponds to the length of the connector 190, so that the cable 180 at the tail end of the connector 190 corresponds to the restraint portion 201 when the connector 190 is inserted into the probe socket 141.

For example, as shown in FIGS. 4-6, the restraint portion 201 is disposed above the probe socket 141 and spaced apart from the probe socket 141 by a certain distance, which is approximately equal to the length of the connector 190, so that when a front end 1901 of the connector 190 is inserted into the probe socket 141, the tail portion 1902 of the connector 190 can open the opening 2011 of the restraint portion 201, and part of the cable 180 connected to the tail portion 1902 can enter the through hole 2010.

In some embodiments, the driving portion 202 of the cable management apparatus 100 is disposed on the base 140, and the base 140 is further provided with a slot portion for the restraint portion 202 of the cable management apparatus 100 to move along the first direction or the second direction.

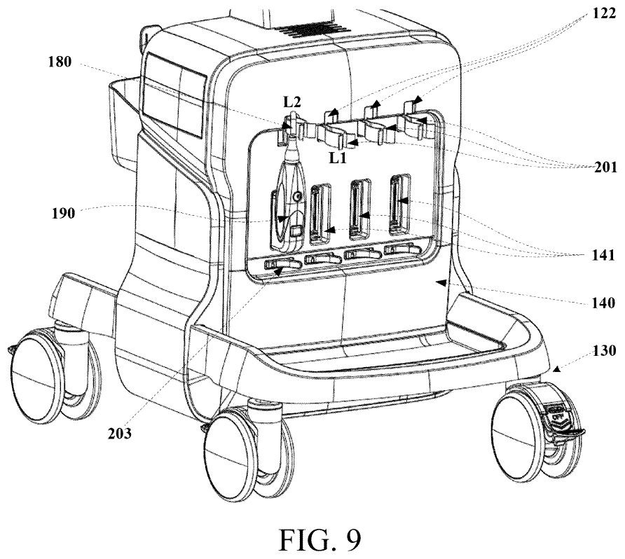

FIG. 9 is a schematic diagram of part of a base according to an embodiment of the present application. For example, as shown in FIG. 9, the base 140 is further provided with a slot portion 122 for the restraint portion 201 of the cable management apparatus 100 to move along the first direction or the second direction. The restraint portion 201 may be movable between the first position L1 and the second position L2 by means of the slot portion 122.

For example, FIG. 9 exemplarily illustrates four restraint portions 201, the leftmost restraint portion 201 is in a use state, the cable 180 has been subjected to cable management, and the connector 190 of the cable 180 has been inserted into the corresponding probe socket 141. As shown in FIG. 9, the leftmost restraint portion 201 has been moved from the first position L1 to the second position L2 via the slot portion 122. The other three restraint portions 201 are in an unused state and are still located at the first position L1.

In some embodiments, the base 140 is further provided with a locking switch 203 for locking the connector 190. The locking switch 203 may further be used as a trigger portion to trigger the driving portion 202 of the cable management apparatus 100.

For example, as shown in FIG. 9, when the locking switch 203 is at a locked position (for example, after the user inserts the connector 190 into the probe socket 141, the user toggles the locking switch 203 to the locked position), the connector 190 is locked within the probe socket 141, and the driving portion 202 is triggered to control the restraint portion 201 to move along the first direction. For another example, when the locking switch 203 is at an unlocked position (for example, the user toggles the locking switch 203 to the unlocked position and prepares to pull out the connector 190 from the probe socket 141), the connector 190 is unlocked from the probe socket 141, and the driving portion 202 is triggered to control the restraint portion 201 to move along the second direction.

In some embodiments, the trigger portion of the cable management apparatus 100 is disposed on the restraint portion 201. When at least part of the cable enters the through hole of the restraint portion, the driving portion is triggered to control the restraint portion to move along the first direction, and/or, when at least part of the cable leaves the through hole of the restraint portion, the driving portion is triggered to control the restraint portion to move along the second direction.

For example, a pressure sensor may be provided at the opening 2011, and the pressure sensor may function as the trigger portion. For example, when the at least part of the cable enters the through hole 2010 of the restraint portion 201, the pressure sensor detects the pressure and sends a first trigger command, and the driving portion 202 is triggered to control the restraint portion 201 to move along the first direction. For another example, when the at least part of the cable leaves the through hole 2010 of the restraint portion 201, the pressure sensor detects the pressure and sends a second trigger command, and the driving portion 202 is triggered to control the restraint portion 201 to move along the second direction. The present application is not limited thereto, and for example, implementation may be performed by means of an actuation switch or a photoelectric switch, etc.

In some embodiments, the trigger portion of the cable management apparatus 100 is disposed in the probe socket. When the connector is inserted into the probe socket, the driving portion is triggered to control the restraint portion to move along the first direction, and/or, when the connector is pulled out from the probe socket, the driving portion is triggered to control the restraint portion to move along the second direction.

For example, a pressure sensor may be provided at the probe socket 141, and the pressure sensor may function as the trigger portion. For example, when the connector 190 is inserted into the probe socket 141, the pressure sensor detects the pressure and sends a first trigger command, and the driving portion 202 is triggered to control the restraint portion 201 to move along the first direction. For another example, when the connector 190 is pulled out from the probe socket 141, the pressure sensor detects the pressure and sends a second trigger command, and the driving portion 202 is triggered to control the restraint portion 201 to move along the second direction. The present application is not limited thereto, and for example, implementation may be performed by means of an actuation switch or a photoelectric switch, etc.

Some cases of the trigger portion are schematically described above, and the present application is not limited thereto, for example, the trigger portion may use a contact component such as a paddle, a button, or a switch, or may use a non-contact component such as photoelectric or magnetic induction components. The trigger portion sends a trigger command to the driving portion when being triggered, and the trigger command may be an ON/OFF signal, or a high/low level, or other control information that can be recognized by the driving portion, which is not limited in the present application.

Although some embodiments of the present application are described on the basis of the medical device having an ultrasonic probe shown in FIG. 1, the embodiments of the present application are not limited thereto. For example, the probe of the medical device may alternatively be a non-ultrasonic probe or the like, and the medical device is not limited to an ultrasonic device. Examples are not listed herein one by one again. The medical device may further include other structural components not shown in FIGS. 1 to 8. The embodiments of the present application are not limited thereto. Specific reference can be made to the related art, which will not be described here again.

According to the above embodiments, the cable management apparatus is disposed in the medical device. The cable management apparatus includes: a restraint portion, having a through hole capable of receiving at least part of a cable of the medical device; and a driving portion, connected to the restraint portion and configured to be triggered to: control the restraint portion to move along a first direction to restrain the at least part of the cable entering the through hole; and/or control the restraint portion to move along a second direction opposite to the first direction. Therefore, the cable can be automatically arranged, thereby omitting the step of manual cable management, so that not only work efficiency can be improved, but user experience can also be improved.

Further, a side surface of the restraint portion is provided with an opening, so that the cable can enter the through hole of the cable management apparatus when the connector of the cable is inserted into the probe socket on the base, thereby further simplifying the operation, reducing human labor, and improving efficiency.

Further, by means of the clamping structure formed by the elastic sheets, the cable enters or leaves the restraint portion by means of elastic deformation, thereby further simplifying the operation and achieving cable management restraint with a simple structure.

Further, the restraint portion is driven to move by means of the motor and the trigger portion, so that the degree of automation of cable management can be improved.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application. As used herein, the term “exemplary” means serving as a non-limiting example, instance, or illustration. As used herein, the term “for example” introduces a list of one or more non-limiting examples, instances, or illustrations.

Preferred implementations of the present application are described above with reference to the accompanying drawings. Many features and advantages of the implementations are clear according to the detailed description. Therefore, the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

1. A cable management apparatus for a medical device, characterized by comprising:
a restraint portion, having a through hole configured to receive at least part of a cable of the medical device; and
a driving portion, connected to the restraint portion and configured to be triggered to:
control the restraint portion to move along a first direction to restrain the at least part of the cable entering the through hole, and/or control the restraint portion to move along a second direction opposite to the first direction.

2. The cable management apparatus according to claim 1, wherein,
the through hole passes through the restraint portion along the first direction and the second direction; and
the restraint portion is further provided with an opening facing a third direction, the at least part of the cable enters or leaves the through hole by means of the opening, and the third direction is perpendicular to the first direction and the second direction.

3. The cable management apparatus according to claim 2, wherein,
the opening has a size smaller than the diameter of the cable, and the cable enters the through hole or leaves the through hole via the opening by means of elastic deformation of the restraint portion.

4. The cable management apparatus according to claim 2, wherein,
the restraint portion has a clamping structure, the clamping structure comprises a first elastic sheet and a second elastic sheet disposed oppositely, and the through hole and the opening are formed between the first elastic sheet and the second elastic sheet.

5. The cable management apparatus according to claim 1, wherein the driving portion comprises:
a motor, configured to drive the restraint portion to move from a first position to a second position along the first direction when receiving a first trigger command, and/or drive the restraint portion to move from the second position to the first position along the second direction when receiving a second trigger command.

6. The cable management apparatus according to claim 5, wherein the cable management apparatus further comprises:
a trigger portion, configured to generate the first trigger command when the at least part of the cable enters the through hole and generate the second trigger command when the at least part of the cable leaves the through hole.

7. A medical device, characterized by comprising:
a probe, comprising a transducer, a cable, and a connector, the cable connecting the transducer and the connector;
a base, comprising a probe socket, the probe socket being capable of receiving the connector; and
the cable management apparatus according to claim 1.

8. The medical device according to claim 7, wherein,
a restraint portion of the cable management apparatus is disposed on the base, the probe socket of the base and the restraint portion are provided along a first direction, and the distance between the probe socket and the restraint portion corresponds to the length of the connector, to enable the cable at the tail end of the connector to correspond to the restraint portion when the connector is inserted into the probe socket.

9. The medical device according to claim 7, wherein,
a driving portion of the cable management apparatus is disposed on the base, and the base is further provided with a slot portion for the restraint portion of the cable management apparatus to move along a first direction or a second direction.

10. The medical device according to claim 7, wherein,
the base is further provided with a locking switch for locking the connector; and the locking switch further serves as a trigger portion for triggering a driving portion of the cable management apparatus.

11. The medical device according to claim 10, wherein,
when the locking switch is in a locked position, the connector is locked within the probe socket, and the driving portion is triggered to control the restraint portion to move along a first direction;
and/or, when the locking switch is in an unlocked position, the connector is unlocked from the probe socket, and the driving portion is triggered to control the restraint portion to move along a second direction.

12. The medical device according to claim 7, wherein,
a trigger portion of the cable management apparatus is disposed on the restraint portion.

13. The medical device according to claim 12, wherein,
when at least part of the cable enters the through hole of the restraint portion, the driving portion is triggered to control the restraint portion to move along a first direction;
and/or, when at least part of the cable leaves the through hole of the restraint portion, the driving portion is triggered to control the restraint portion to move along a second direction.

14. The medical device according to claim 7, wherein,
a trigger portion of the cable management apparatus is disposed in the probe socket.

15. The medical device according to claim 14, wherein,
when the connector is inserted into the probe socket, the driving portion is triggered to control the restraint portion to move along a first direction;
and/or, when the connector is pulled out from the probe socket, the driving portion is triggered to control the restraint portion to move along a second direction.
